(19) United States
(12) Patent Application Publication
(10) Pub. No.: US 2025/0090629 A1
(43) Pub. Date: Mar. 20, 2025

(54) ALLEVIATING DRUG NEPHROTOXICITY

(71) Applicant: The Trustees of Indiana University, Bloomington, IN (US)

(72) Inventors: Bruce A. Molitoris, Centennial, CO (US); Mark C. Wagner, Indianapolis, IN (US); Ruben M. Sandoval, JR., Indianapolis, IN (US)

(73) Assignee: The Trustees of Indiana University, Bloomington, IN (US)

(21) Appl. No.: 18/293,903

(22) PCT Filed: Jul. 28, 2022
(86) PCT No.: PCT/US2022/038616

§ 371 Date: Jan. 31, 2024

Related U.S. Application Data
(60) Provisional application No. 63/337,798, filed on May 3, 2022.
(60) Provisional application No. 63/228,673, filed on Aug. 3, 2021.

(51) Int. Cl. A61K 38/17; A61K 9/00; A61K 31/7036; A61P 39/00

(52) U.S. Cl. CPC A61K 38/1709; A61K 9/0019; A61K 31/7036; A61P 39/00

(57) ABSTRACT

Aspects of the present disclosure relate to novel medical compositions and methods of using same. In certain aspects, the disclosure relates to methods preventing acute kidney injury in a patient associated with increased amount of protein in the urine (e.g. albuminuria). In certain embodiments, the disclosure provides methods of preventing or treating acute kidney injury in a patient receiving a nephrotoxic drug, by administration of a reversible endocytosis inhibitor.

This invention was made with government support under DK091623 awarded by National Institutes of Health. The government has certain rights in the invention.

### BACKGROUND

Nephrotoxicity from many different drugs is responsible for twenty percent of acute kidney injury. This results in hospitalization, increased length of stay, morbidity, and mortality. Many therapeutic agents, including antibiotics such as aminoglycosides, are filtered by the kidney and are reabsorbed by the proximal tubule. Many of these nephrotoxic drugs cause injury to the proximal tubule following endocytosis from the glomerular filtrate. The proximal tubule reabsorbs and concentrates filtered nephrotoxins by clathrin mediated and fluid phase mediated endocytosis, leading to proximal tubule injury and acute kidney injury. Thus, a need exists for improved methods of preventing kidney injury during administration of nephrotoxic drugs.

### SUMMARY

In certain aspects, the present disclosure provides unique medical compositions and methods of using same. In accordance with some forms, such medical compositions are useful for preventing acute kidney injury in a patient being treated with a nephrotoxic drug.

Accordingly, in one embodiment, the present disclosure provides a method of preventing kidney injury in a patient taking a nephrotoxic drug, the method comprising administering an endocytosis inhibitor to the patient. In certain embodiments, the endocytosis inhibitor comprises Receptor Associated Protein (RAP), which may include human, mouse, or rat RAP. In some forms, the RAP is administered at a dose of 5-100 mg/Kg, optionally 10-60 mg per kg patient body weight. In accordance with certain inventive variants, the endocytosis inhibitor is administered intravenously. In some forms, the method further comprises treating the patient with a nephrotoxic drug, for example an aminoglycoside such as gentamicin. In certain embodiments, the endocytosis inhibitor is administered prior to treatment with the nephrotoxic drug. In certain embodiments, the endocytosis inhibitor is administered concurrently with treatment with the nephrotoxic drug.

In another embodiment, the present disclosure provides receptor-associated protein (RAP) for use in preventing acute kidney injury. In certain embodiments, the RAP is provided for use in preventing acute kidney injury in a patient taking a nephrotoxic drug, such as an aminoglycoside, for example gentamicin. In accordance with some forms, the RAP provided comprises alpha-2-macroglobulin RAP.

In another embodiment, the present disclosure provides for use of receptor-associated protein (RAP) for the manufacture of a medicament to prevent acute kidney injury. In certain embodiments, the RAP comprises alpha-2-macroglobulin RAP, and may comprise human, rat, or mouse RAP. In accordance with some forms, the medicament is configured for intravenous administration.

In yet another embodiment, the present disclosure provides a pharmaceutical composition for the prevention of acute kidney injury in a patient taking a nephrotoxic drug, the composition comprising receptor-associated protein (RAP). In certain embodiments, the pharmaceutical composition comprises alpha-2-macroglobulin RAP. In some forms, the pharmaceutical composition is configured for intravenous administration. In accordance with certain inventive variants, the pharmaceutical composition is configured for administration at a dose of 10-60 mg per kilogram of patient body weight.

In another embodiment, the present disclosure provides a method of measuring glomerular function, the method comprising administering an endocytosis inhibitor to the patient, obtaining a urine sample from the patient, after said administering, and measuring the amount of protein in the urine sample. In certain embodiments, the endocytosis inhibitor comprises Receptor Associated Protein (RAP), which may include human, mouse, or rat RAP. In some forms, the RAP is administered at a dose of 5-100 mg/Kg, optionally 10-60 mg per kg patient body weight. In accordance with certain inventive variants, the endocytosis inhibitor is administered intravenously. In certain embodiments the protein measured is albumin. In some forms the method also comprises obtaining an initial urine sample prior to administering an endocytosis inhibitor to the patient, and measuring the amount of protein in the urine sample, for example in some forms an amount of protein produced over a time period is measured, and/or as a ratio of urinary albumin to urinary creatinine is determined. Further forms, objects, features, aspects, benefits, advantages, and embodiments of the present invention will become apparent from a detailed description and drawings provided herewith.

### BRIEF DESCRIPTION OF THE DRAWINGS

FIG. 1 is a graphical representation of the results of Example 1.

FIG. 2 is a graphical representation of the results of Example 2.

FIG. 3 is a graphical representation of the results of Example 3.

FIG. 4A-B shows single plane images of vehicle treated or RAP treated rats as described in Example 5.

FIG. 4C-F are graphical representations of the results of Example 5.

### DESCRIPTION OF THE SELECTED EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. One embodiment of the invention is shown in great detail, although it will be apparent to those skilled in the relevant art that some features that are not relevant to the present invention may not be shown for the sake of clarity.

As disclosed above, aspects of the present disclosure relate to novel medical compositions and methods of using same. In certain aspects, the disclosure relates to methods preventing acute kidney injury in a patient. In certain embodiments, the disclosure provides methods of preventing or treating acute kidney injury in a patient receiving a nephrotoxic drug, by administration of an endocytosis inhibitor as described herein.

In accordance with certain embodiments, the present disclosure provides pharmaceutical compositions and uses of same that inhibit endocytosis of one or more exogenous and endogenous nephrotoxic agents. Applicants have surprisingly discovered that inhibition of certain membrane proteins found in the proximal tubules is advantageous to preventing acute kidney injury by nephrotoxic therapeutics being administered to a patient. The inhibition may include membrane proteins particularly in the S1 region of the proximal tubule, and may include other proximal tubule segments such as S2 and S3. Without wishing to be bound by any mechanism, Applicants submit that the inhibition of membrane proteins Megalin and/or Cubilin sufficiently inhibits endocytic uptake of harmful nephrotoxic agents. It has been discovered that the use of such endocytosis inhibitors prevents uptake of nephrotoxic therapeutics by inhibiting fluid phase and receptor mediated endocytosis of the nephrotoxin, thus preventing accumulation of nephrotoxic therapeutic and subsequent acute kidney injury while the nephrotoxic therapeutic is present in the body.

The compositions and methods of the present disclosure may also inhibit uptake of certain endogenous proteins known to be nephrotoxic and endocytosed by the proximal tubule after filtration, for example myoglobin, hemoglobin, Danger Associated Molecular Patterns (DAMPs), and/or Pathogen Associated Molecular Patterns (PAMPs). Myoglobin is released with muscle injury and causes rhabdomyolysis and acute kidney injury. Free hemoglobin is released during hemolysis and causes acute kidney injury. DAMPs are proteins released from injured cells. PAMPS are proteins released by infectious agents. DAMPs and PAMPs cause injury to proximal tubule cells.

In certain embodiments, the compositions and methods of the present disclosure can be useful for preventing kidney injury caused by one or more exogenous and/or endogenous nephrotoxins. Such nephrotoxins may comprise a nephrotoxic therapeutic, radiopharmaceuticals, and/or radiocontrast composition. In accordance with some forms, the disclosed compositions are effective to prevent kidney injury caused by antibiotics, preferably aminoglycosides. Thus, in accordance with certain embodiments, a patient being treated with one or more aminoglycosides may be administered an endocytosis inhibitor as disclosed herein. Thus, certain methods of the present disclosure may comprise the step of administering one or more nephrotoxic therapeutics, for example an aminoglycoside. The nephrotoxic therapeutic, or drug, may be administered in any suitable manner, for example intraperitoneal or intravenous injection. As disclosed herein the term aminoglycoside may include one or more of the following: gentamicin, amikacin, tobramycin, kanamycin, streptomycin, and/or neomycin. In certain preferred embodiments, the methods of the present disclosure include methods of preventing kidney injury caused by the administration of gentamicin to a patient.

In accordance with certain embodiments, the present disclosure provides compositions and methods comprising an endocytosis inhibitor. In certain embodiments, the endocytosis inhibitor comprises Alpha-2-macroglobulin receptor-associated protein (Alpha-2-MRAP) also known as low-density lipoprotein receptor-related protein-associated protein 1, also known as Receptor Associated Protein (RAP). In some forms, the RAP used in the present disclosure is a stable mutant RAP comprising Alpha-2-macroglobulin RAP. See, Joni M. Prasad, Mary Migliorini, Rebeca Galisteo, and Dudley K. Strickland, Generation of a Potent Low-Density Lipoprotein Receptor-related Protein 1 (LRP1) Antagonist by Engineering a Stable Form of the Receptor-associated Protein (RAP) D3 Domain, J Biol Chem, 2015 Jul. 10; 290 (28): 17262-17268, incorporated herein by reference describes a 5 amino acid change which results in a more stable association of RAP with the receptor at low pH, thus prolonging ligand inhibition. RAP is a 39 kD protein that acts as a molecular chaperone for LDL receptor-related proteins, for example Megalin, and regulates their binding activity along the secretory pathway. When bound RAP temporarily prevents other ligands, such as nephrotoxins, from binding receptor proteins. Any suitable RAP may be used as disclosed herein, for example, RAP sequences from human, rat, and mouse sources are provided herein as SEQ ID NO 1 (human), SEQ ID NO 2 (rat), and SEQ ID NO 3 (mouse). Applicants have surprisingly found that inhibition of endocytosis by RAP is temporary and reversible without causing lasting effects on kidney function. Thus, the present disclosure provides compositions and methods which temporarily inhibit endocytosis which may provide advantageous use, such as immediately prior to and during administration of nephrotoxic therapeutics or other substances as disclosed herein.

The endocytosis inhibitor of the present disclosure may be administered in any suitable manner. Applicants have discovered that intravenous administration is particularly advantageous as the endocytosis inhibitor is delivered immediately to the bloodstream. In particular embodiments utilizing RAP a dose of 5 mg to 100 mg per kg of patient body weight is administered, preferably 10 mg to 60 mg per kg of patient body weight, more preferably 30 mg to 50 mg per kg of patient body weight. In accordance with some forms, about 40 mg per kg of patient body weight is administered.

In some forms, the endocytosis inhibitor is administered prior to treating the patient with the nephrotoxic drug. For example, a patient may be receiving an intravenous administration of RAP prior to treatment with gentamicin. It has been discovered that the therapeutic effects on endocytosis (i.e. prevention of kidney uptake and injury) are reversible and last for at least 1 to 2 hours after which time the normal kidney function is rapidly resumed. Thus, in accordance with some forms, a continuous infusion of the endocytosis inhibitor is administered while a patient is undergoing treatment with a nephrotoxic drug.

As discussed herein, aspects of the present disclosure also relate to novel methods of detecting disease or damage to the kidney, in particular glomerular disease or injury which may affect the ability of the glomeruli to correctly filter blood. Glomerular disease is associated with increased amount of protein in the urine (e.g. albuminuria) due to ineffective filtering of wastes from the blood. A number of different diseases can result in glomerular disease or injury. It may be the direct result of an infection or a drug toxic to the kidneys, or it may result from a disease that affects the entire body, like diabetes or Systemic Lupus Erythematosus. Many different kinds of diseases can cause swelling or scarring of the nephron or glomerulus. Thus, the methods of the present disclosure may be used to detect glomerular disease or injury associated with one or more of the following diseases or conditions: systemie lupus erythematosus, anti-GBM disease, IgA nephropathy, hereditary nephritis (Alport syndrome), acute post-streptococcal glomerulonephritis, bacterial endocarditis, HIV, glomerulosclerosis, diabetic nephropathy, focal segmental glomerulosclerosis, membranous glomerulopathy, and or chronic kidney disease.

Applicants have discovered a novel method for assessing glomerular function which allows for early stage detection of glomerular disease or injury. Applicants have discovered that in certain cases excess protein filtered in the glomerulus is reabsorbed by the proximal tubule. The reabsorption of filtered protein (e.g. albumin) would not be detected by a traditional urinalysis. Applicants have developed a method which includes inhibiting protein uptake in the proximal tubule and measuring the amounts of protein in the urine. In this way, excessive protein levels being filtered from the glomerulus may be detected before significant harm is caused. In certain embodiments the protein comprises albumin. In certain embodiments, inhibiting protein uptake comprises administration of RAP as disclosed above. It may be useful to record a baseline level of protein in the urine prior to administration of the uptake inhibitor in order to calculate the amount of protein being reabsorbed by the proximal tubule.

To promote a further understanding of embodiments disclosed herein and their features and advantages, the following specific Examples are provided. It will be understood that these examples are illustrative and not limiting in nature.

### Example 1

### RAP Protects Against Gentamicin Induced Nephrotoxicity

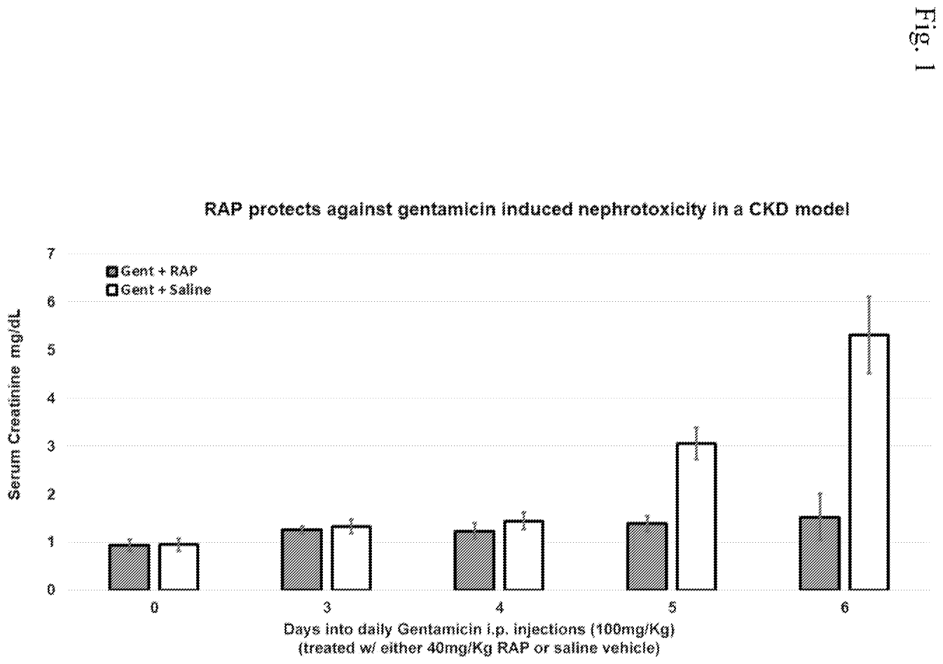

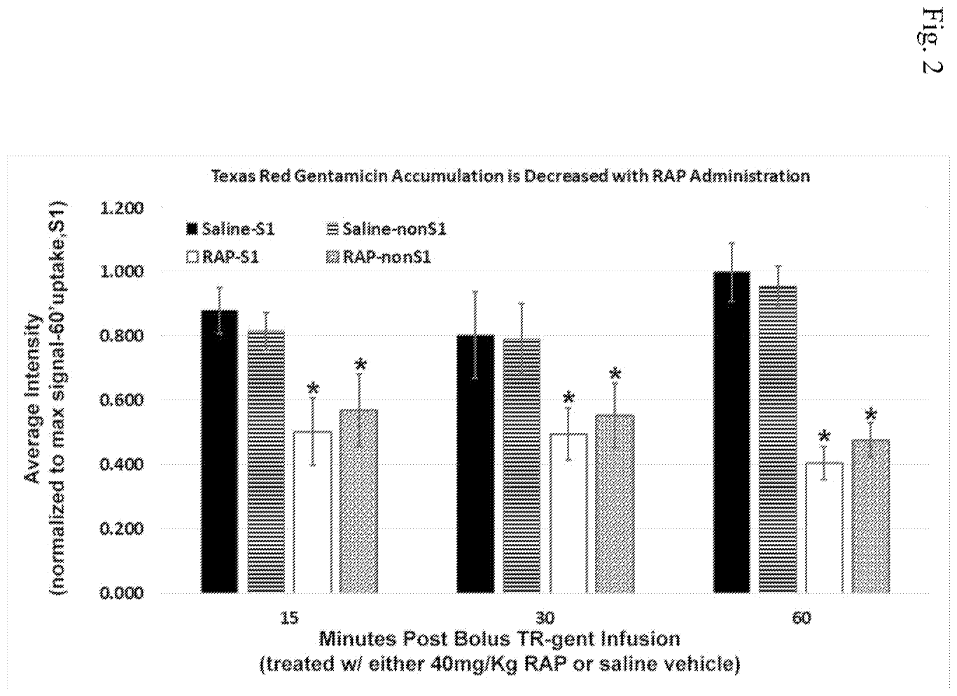

Rats with a Chronic Kidney Disease (CKD) background were given either saline or 40 mg/kg RAP. Both group were then given an intraperitoneal injection of gentamicin (100 mg/kg) daily for five days. Serum creatinine, an indicator of renal function, was measured in both rat populations at day 0, 3, 4, 5, and 6. The results, illustrated in FIG. 1, show an increase in serum creatinine, indicating a decrease in kidney function, in those rats given the saline vehicle. Conversely, rats given RAP and gentamicin did not exhibit the same increase in serum creatinine.

### Example 2

Texas Red Gentamicin Accumulation is Decreased with RAP Administration.

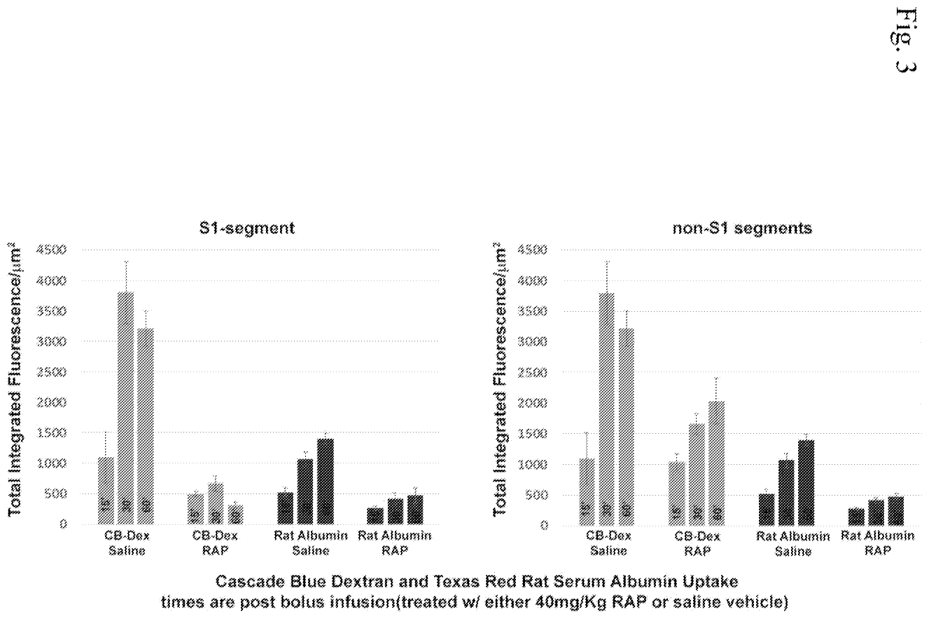

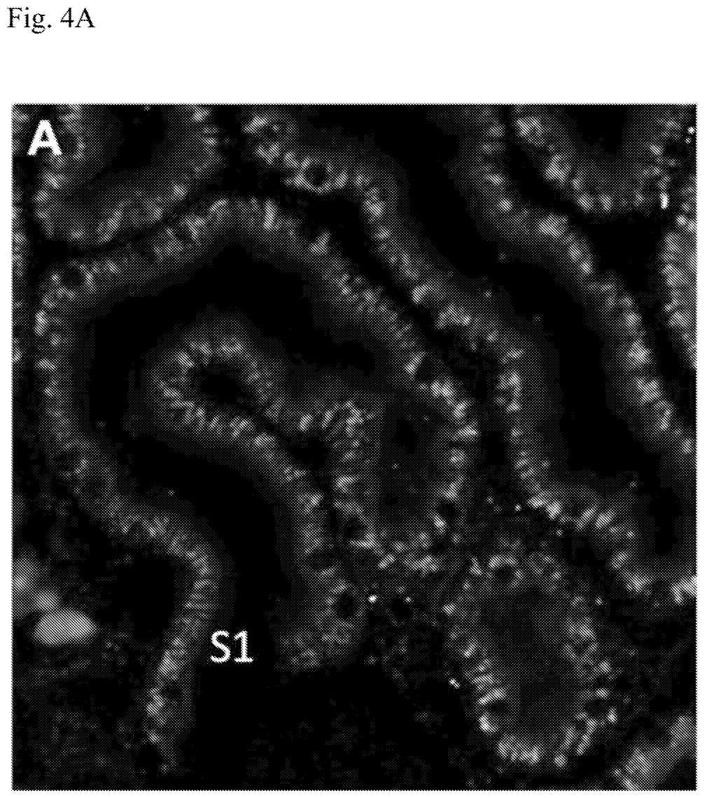

Rats were given either RAP (40 mg/kg) or a saline vehicle followed by a single bolus of Texas Red Gentamicin. Intra-vital 2-photon images of both populations were quantified to determine average fluorescence intensity within S1 and non-S1 proximal tubule segments. The values for the saline vehicle treated rats were consistently significantly higher than the RAP treated rats in both the S1 segment and non-S1 segments of the proximal tubules during the progression of internalization and accumulation. The results of this experiment are illustrated in FIG. 2.

### Example 3

### Cascade Blue Dextran and Texas Red Rat Serum Albumin Uptake Times Post Bolus Infusion (RAP or Saline).

Rats were given either RAP or saline vehicle followed by a bolus containing a fluid phase endocytic marker (10,000 MW Cascade Blue Dextra, CB-dex) and a receptor mediated uptake marker (Texas Red Rat Serum Albumin, Rat Albumin). Both markers showed reduction in proximal tubule uptake and accumulation across the 15′, 30′, and 60′ post bolus uptake times, in both S1 and non-S1 proximal tubule segments. The results of this experiment are illustrated in FIG. 3.

### Example 4

We utilized daily injections of gentamicin (100 mg/kg, intraperitoneal) in a uninephrectomy chronic kidney disease model in Munich Wister Fromter rats with a baseline serum creatinine (sCr) of 0.80±0.23. Gentamicin was given with or without RAP (40 mg/kg, intravenous) to evaluate RAP's impact on function, sCr, 24-hour urinary creatinine clearance and proteinurea, and endocytosis, 2-photon microscopy to determine fluid phase mediated endocytosis i.e. 10 kDa Cascade Blue dextran, and Megalin mediated clathrin mediated endocytosis, i.e. albumin, endocytosis.

It was discovered that RAP injections markedly reduced S1 proximal tubule albumin uptake over 60 minutes (80%), dextran (67%), and gentamicin (62%) in normal rats in a rapid fully reversible fashion. In rats treated with or without RAP, daily gentamicin treatment resulted in elevated serum Cr by day 5 (1.4±0.5 vs 3.1±0.4 mg/dl, p<0.01) and day 6 (1.5±0.5 vs 5.4±0.8 mg/dl, p<0.01). 24-hour creatinine clearance, a measure of glomerular filtration rate (kidney functions) decreased from a CKD pretreatment baseline of 0.91=0.22 ml/min to 0.49±0.16 ml/min and 0.09±0.07 ml/min and urinary protein increased from a CKD pretreatment baseline of 221±40 mg/ml/100 μm wt to 488 mg/ml/100 μm wt vs 2,512 mg/ml/100 μm wt in RAP treated and untreated rats, respectively.

These results indicate RAP treatment induced reductions of both receptor mediated endocytosis and fluid phase endocytosis suggesting a link between Megalin and fluid phase endocytosis. Clinically, RAP may have direct relevance to preventing the harmful nephrotoxic effects of gentamicin treatment, and likely other nephrotoxins, especially in individuals more susceptible to aminoglycoside or nephrotoxic injury.

### Example 5

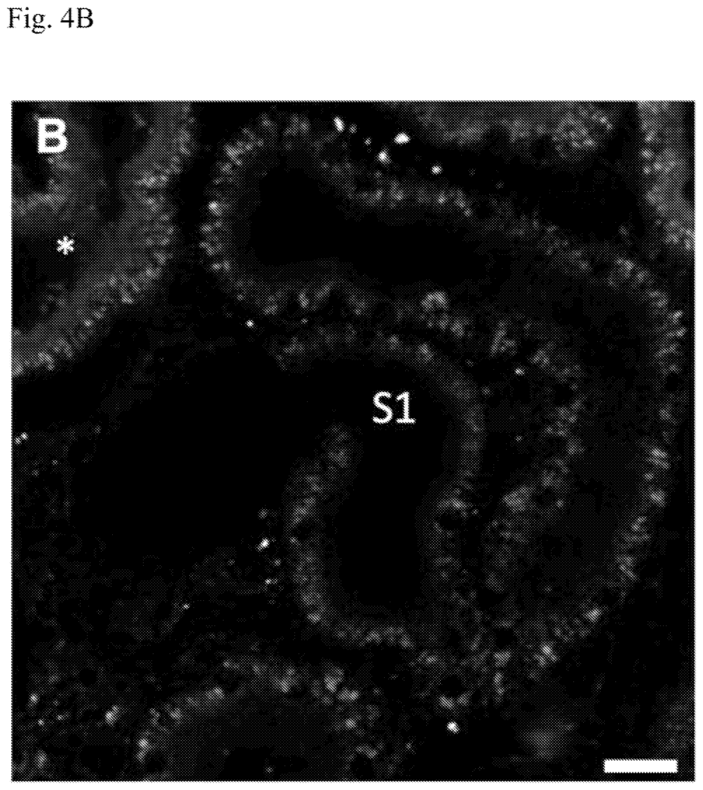

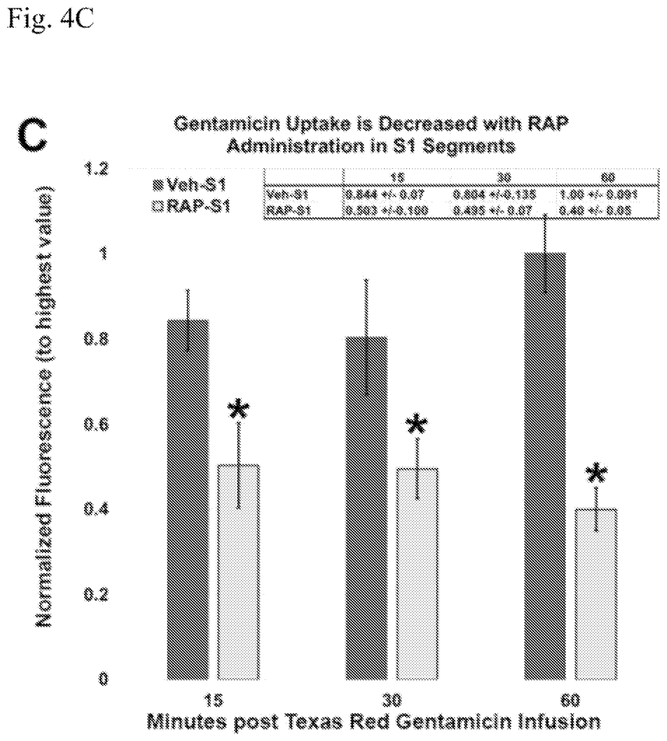

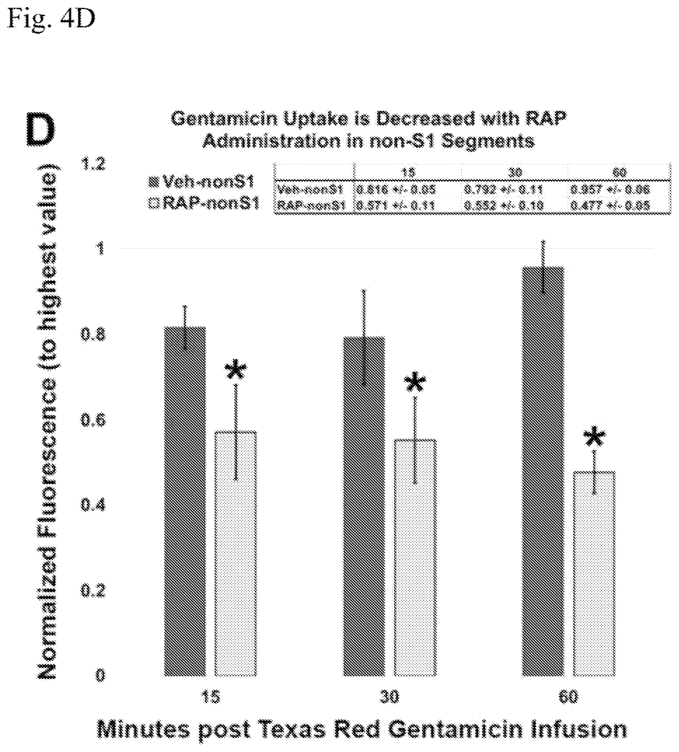

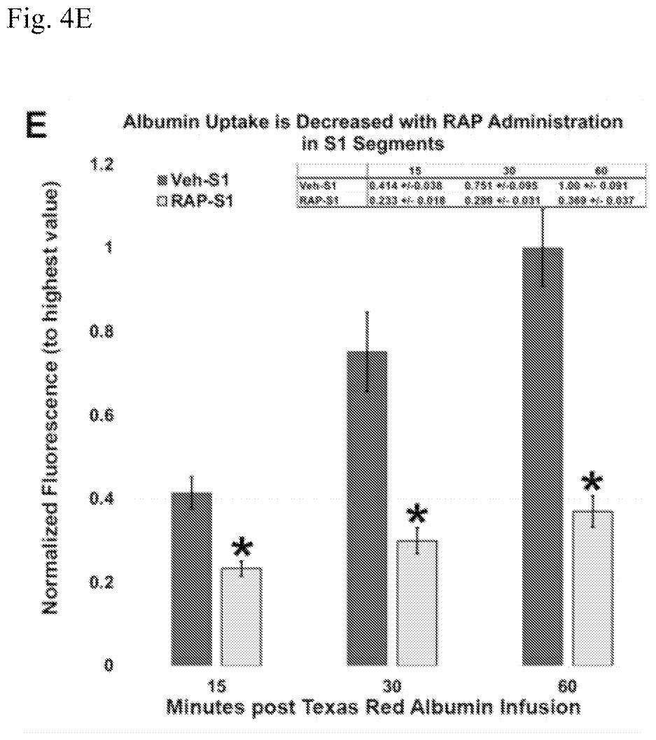

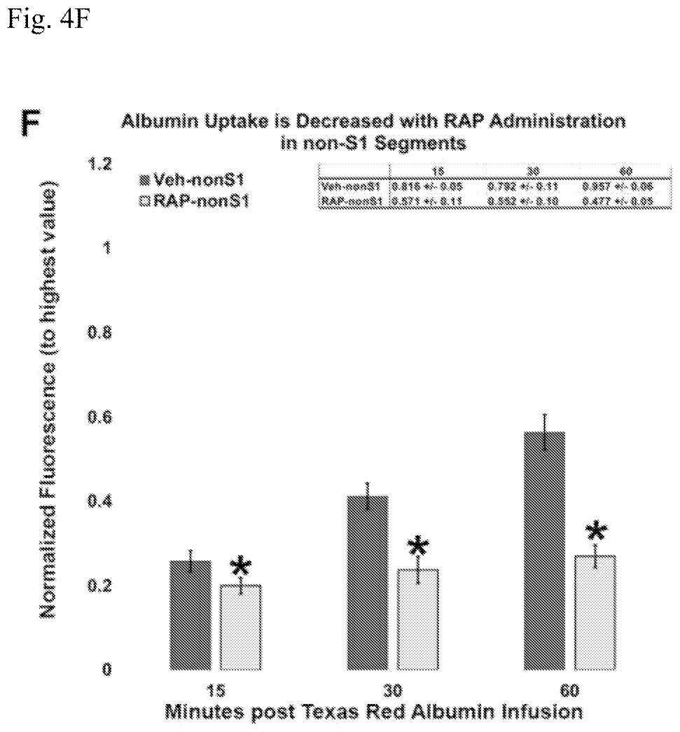

Rats were infused with either vehicle control or non-fluorescent RAP at 40 mg/Kg. Ten minutes later, a bolus of Texas Red rat serum albumin (TR-RSA) and 10 kDa Cascade Blue dextran was given. FIG. 4A shows single plane images of vehicle treated rats 60 minutes post initial bolus infusion. FIG. 4B shows single plane images of RAP treated rats 60 minutes post initial bolus infusion. In FIG. 4A, the S1 segment in the vehicle treated rat accumulated more TR-RSA (red) and 10 kDa dextran (blue) producing a magenta color in the 10 endocytic pathway. In contract, the RAP treated rates (FIG. 4B) show virtually no uptake of the TR-RSA (red), and only faint accumulation of the 10 kDa dextran (blue), in small endocytic vesicles within the S1 segment (G indicated glomerulus). Images collected from these studies at approximately 15, 30, and 60 minutes post initial infusion were quantified for TR-RSA and 10 kDa dextran uptake in either vehicle or RAP treated rats, in both S1 and non-S1 proximal tubule segments. Values were reported as normalized fluorescence, with the values normalized to the highest average value in the study group. FIGS. 4C and 4D show the differences in uptake between the vehicle and RAP treated rats in S1 (FIG. 4C) and non-S1 (FIG. 4D) proximal tubule segments for the 10 kDa Cascade Blue dextran. The differences between vehicle and RAP treated were significant at all three time points studied and in both the S1 and non-S1 proximal tubule segments. The rapid filtration and avid uptake of the Cascade Blue dextran bolus shows the greater disparity between the vehicle untreated (Veh) group and the RAP treated (RAP) group; as compared to the albumin uptake data. Here, the reduction of dextran uptake in RAP treated rats is well over 5-fold in the S1-segment (FIG. 4C); this disparity is lessened in non-S1 proximal tubule segments to a 2-fold decrease in RAP treated rats (FIG. 4D), FIGS. 4E and 4F show the effects of RAP on Texas Red albumin accumulation in S1 (FIG. 4B) and non-S1 (FIG. 4F) proximal tubule segments. The lower glomerular sieving coefficient of the RSA results in less RSA filtration and this a slower, protracted uptake that appears linear over time, with the S1 segment having the greatest accumulation. RAP treated rats had significantly reduced RSA uptake at all the time points studied and in both S1 (FIG. 4E) and non-$1 (FIG. 4F) proximal tubule segments when compared to vehicle treated rats. In FIGS. 4C-4F tables are inserted to show the normalized values+/−standard error. (Bar=20 μm).

### Embodiments

The following provides an enumerated listing of some of the embodiments disclosed herein. It will be understood that this listing is non-limiting, and that individual features or combinations of features (e.g. 2, 3 or 4 features) as described in the Detailed Description above can be incorporated with the below-listed Embodiments to provide additional disclosed embodiments herein

1. A method of preventing acute kidney injury in a patient taking a nephrotoxic drug, the method comprising:
administering an endocytosis inhibitor to the patient.

2. The method of embodiment 1, wherein the endocytosis inhibitor comprises Receptor Associated Protein (RAP).

3. The method of embodiment 2, wherein the RAP comprises human RAP.

4. The method of embodiment 3, wherein the human RAP comprises Human alpha-2-macroglobulin RAP.

5. The method of any one of embodiments 2 through 4, wherein the RAP comprises murine RAP.

6. The method of any one of embodiments 2 through 5, wherein the RAP is administered at a dose of 5-100 mg per kg patient body weight.

7. The method of any one of the preceding embodiments, wherein said administering comprises intravenous administration of the endocytosis inhibitor to the patient.

8. The method of any one of the preceding embodiments, further comprising:
treating a patient with a nephrotoxic drug.

9. The method of embodiment 8, wherein said administering occurs prior to said treating.

10. The method of any one of embodiment 8 or 9, wherein said administering occurs concurrent with said treating.

11. The method of any one of the preceding embodiments, wherein the nephrotoxic drug comprises a therapeutic molecule that is filtered at the proximal tubule.

12. The method of any one of the preceding embodiments, wherein the nephrotoxic drug comprises an aminoglycoside.

13. The method of any one of the preceding embodiments, wherein the nephrotoxic drug comprises gentamicin.

14. Receptor-associated protein (RAP) for use in preventing acute kidney injury.

15. The RAP of embodiment 14, for use in preventing acute kidney injury in a patient taking a nephrotoxic drug.

16. The RAP of embodiment 15, wherein the nephrotoxic drug comprises an aminoglycoside.

17. The RAP of embodiment 16, wherein the aminoglycoside comprises gentamicin.

18. The RAP of any one of the preceding embodiments, wherein the RAP comprises alpha-2-macroglobulin RAP.

19. Use of receptor-associated protein (RAP) for the manufacture of a medicament to prevent acute kidney injury.

20. The use of embodiment 19, wherein the RAP comprises alpha-2-macroglobulin RAP.

21. The use of any one of embodiments 19 or 20, wherein the RAP is human RAP.

22. The use of any one of embodiments 19 through 21, wherein the medicament is configured for intravenous administration.

23. The use of any one of embodiments 19 through 22, wherein the medicament comprises a medicament to prevent acute kidney injury in a patient taking a nephrotoxic drug.

24. The use of embodiment 23, wherein the nephrotoxic drug comprises an aminoglycoside.

25. The use of embodiment 24, wherein the aminoglycoside comprises gentamicin.

26. A pharmaceutical composition for the prevention of acute kidney injury in a patient taking a nephrotoxic drug, the composition comprising receptor-associated protein (RAP).

27. The pharmaceutical composition of embodiment 26, wherein the RAP comprises alpha-2-macroglobulin RAP.

28. The pharmaceutical composition of any one of embodiments 26 or 27, wherein the RAP comprises human RAP.

29. The pharmaceutical composition of any one of embodiments 26 through 28, wherein the RAP is configured for intravenous administration.

30. The pharmaceutical composition of any one of embodiments 26 or 29, wherein the RAP is configured for administration at a dose of 10-100 mg/Kg, optionally 20-60 mg per kilogram, of patient body weight.

31. The pharmaceutical composition of any one of embodiments 26 or 30, wherein the nephrotoxic drug comprises an aminoglycoside.

32. The pharmaceutical composition of embodiment 31, wherein the aminoglycoside comprises gentamicin.

33. A method of measuring glomerular function; the method comprising:
administering an endocytosis inhibitor to the patient;
obtaining a urine sample from the patient, after said administering; and
measuring the amount of protein in the urine sample.

34. The method of embodiment 33, wherein the endocytosis inhibitor comprises Receptor Associated Protein (RAP).

35. The method of embodiment 34, wherein the RAP comprises human RAP.

36. The method of embodiment 35, wherein the human RAP comprises Human alpha-2-macroglobulin RAP. 37. The method of embodiment 34, wherein the RAP comprises murine RAP.

38. The method of any one of embodiments 34 through 37, wherein the RAP is administered at a dose of 5-100 mg per kg patient body weight.

39. The method of any one of embodiments 33 through 38, wherein said administering comprises intravenous administration of the endocytosis inhibitor to the patient.

40. The method of any one of embodiments 33 through 39, wherein said measuring comprises measuring the amount of albumin in the urine sample.

41. The method of any one of embodiments 33 through 40, also comprising:
obtaining an initial urine sample prior to administering an endocytosis inhibitor to the patient; and
measuring the amount of protein in the urine sample.

While the invention has been illustrated and described in detail in the drawings and foregoing description, the same is to be considered as illustrative and not restrictive in character, it being understood that only the preferred embodiment has been shown and described and that all changes, equivalents, and modifications that come within the spirit of the inventions defined by following claims are desired to be protected. All publications, patents, and patent applications cited in this specification are herein incorporated by reference as if each individual publication, patent, or patent application were specifically and individually indicated to be incorporated by reference and set forth in its entirety herein.

What is claimed is:

1. A method of preventing acute kidney injury in a patient taking a nephrotoxic drug, the method comprising:
administering an endocytosis inhibitor to the patient.

2. The method of claim 1, wherein the endocytosis inhibitor comprises Receptor Associated Protein (RAP).

3. The method of claim 2, wherein the RAP comprises human RAP.

4. The method of claim 3, wherein the human RAP comprises Human alpha-2-macroglobulin RAP.

5. The method of claim 2, wherein the RAP comprises murine RAP.

6. The method of claim 2, wherein the RAP is administered at a dose of 5-100 mg per kg patient body weight.

7. The method of claim 1, wherein said administering comprises intravenous administration of the endocytosis inhibitor to the patient.

8. The method of claim 1, further comprising:
treating a patient with a nephrotoxic drug.

9. The method of claim 8, wherein said administering occurs prior to said treating.

10. The method of claim 8, wherein said administering occurs concurrent with said treating.

11. The method of claim 8, wherein the nephrotoxic drug comprises a therapeutic molecule that is filtered at the proximal tubule.

12. The method of claim 11, wherein the nephrotoxic drug comprises an aminoglycoside.

13. The method of claim 12, wherein the nephrotoxic drug comprises gentamicin.

14. Receptor-associated protein (RAP) for use in preventing acute kidney injury.

15. The RAP of claim 14, for use in preventing acute kidney injury in a patient taking a nephrotoxic drug.

16. The RAP of claim 15, wherein the nephrotoxic drug comprises an aminoglycoside.

17. The RAP of claim 16, wherein the aminoglycoside comprises gentamicin.

18. The RAP of claim 14, wherein the RAP comprises alpha-2-macroglobulin RAP.

19. Use of receptor-associated protein (RAP) for the manufacture of a medicament to prevent acute kidney injury.

20. The use of claim 19, wherein the RAP comprises alpha-2-macroglobulin RAP.

21. The use of claim 20, wherein the RAP is human RAP.

22. The use of claim 19, wherein the medicament is configured for intravenous administration.

23. The use of claim 19, wherein the medicament comprises a medicament to prevent acute kidney injury in a patient taking a nephrotoxie drug.

24. The use of claim 23, wherein the nephrotoxic drug comprises an aminoglycoside.

25. The use of claim 24, wherein the aminoglycoside comprises gentamicin.

26. A pharmaceutical composition for the prevention of acute kidney injury in a patient taking a nephrotoxic drug, the composition comprising receptor-associated protein (RAP).

27. The pharmaceutical composition of claim 26, wherein the RAP comprises alpha-2-macroglobulin RAP.

28. The pharmaceutical composition of claim 27, wherein the RAP comprises human RAP.

29. The pharmaceutical composition of claim 26, wherein the RAP is configured for intravenous administration.

30. The pharmaceutical composition of claim 26, wherein the RAP is configured for administration at a dose of 10-100 mg per kilogram of patient body weight.

31. The pharmaceutical composition of claim 26, wherein the nephrotoxic drug comprises an aminoglycoside.

32. The pharmaceutical composition of claim 31, wherein the aminoglycoside comprises gentamicin.

33. A method of measuring glomerular function; the method comprising:
administering an endocytosis inhibitor to the patient;
obtaining a urine sample from the patient, after said administering; and
measuring the amount of protein in the urine sample.

34. The method of claim 33, wherein the endocytosis inhibitor comprises Receptor Associated Protein (RAP).

35. The method of claim 34, wherein the RAP comprises human RAP.

36. The method of claim 35, wherein the human RAP comprises Human alpha-2-macroglobulin RAP.

37. The method of claim 34, wherein the RAP comprises murine RAP.

38. The method of claim 34, wherein the RAP is administered at a dose of 5-100 mg per kg patient body weight.

39. The method of claim 33, wherein said administering comprises intravenous administration of the endocytosis inhibitor to the patient.

40. The method of claim 33, wherein said measuring comprises measuring the amount of albumin in the urine sample.

41. The method of claim 33, also comprising:
obtaining an initial urine sample prior to administering an endocytosis inhibitor to the patient; and
measuring the amount of protein in the urine sample.
